(19) United States
(12) Patent Application Publication
(10) Pub. No.: US 2003/0040049 A1
(43) Pub. Date: Feb. 27, 2003

(54) Mutant lactic bacteria with a capacity for overexpressing at least one peptidase

(75) Inventors: Eric Guedon, Boulogne (FR); Jamila Anba-Mondoloni, Montigny-le-Bretonneux (FR); Christine Delorme, Palaiseau (FR); Pierre Renault, Montigny-le-Bretonneux (FR)

Correspondence Address: SCHNADER HARRISON SEGAL & LEWIS, LLP, 1600 MARKET STREET, SUITE 3600, PHILADELPHIA, PA, 19103

(73) Assignee: Institut National de la Recherche Agronomique-INRA, Paris (FR)

(21) Appl. No.: 10/122,466

(22) Filed: Apr. 15, 2002

Related U.S. Application Data
(63) Continuation of application No. PCT/FR00/02869, filed on Oct. 13, 2000.

(30) Foreign Application Priority Data
Oct. 15, 1999 (FR) 99/12924

(51) Int. Cl. C12P 21/02; C12N 1/21; C12N 15/74

(52) U.S. Cl. 435/69.1; 435/252.3; 435/320.1

(57) ABSTRACT

A mutant lactic bacteria with a capacity for overexpressing one or more peptidases, wherein gene codY is inactivated.

### RELATED APPLICATION

This is a continuation of International Application No. PCT/FR0002869, with an international filing date of Oct. 13, 2000, which is based on French Patent Application No. 99/12924, filed Oct. 15, 1999.

### FIELD OF THE INVENTION

This invention concerns mutants of lactic bacteria, such as Lactococcus lactis, with a capacity for overexpressing at least one and preferably multiple peptidases. These mutants exhibit strong peptidolytic activities which enable acceleration of the degradation of casein in amino acids. These mutants are thus most particularly useful for augmenting the maturation rate of cheeses since the amino acids are precursors in the synthesis of aromas. The invention also concerns a method for identifying these mutants and genetic constructions for implementing this method. Finally, the invention also concerns the use of these mutant bacteria in a cheese fabrication and/or maturation process.

### BACKGROUND

Lactococcus lactis has a complex proteolytic system for degrading milk proteins, especially casein. Casein is the dominant protein in milk and provides the amino acids required for growth [12]. Casein is degraded into oligopeptides by a wall protease. These oligopeptides enter the cell via specific transport systems and then are hydrolyzed into amino acids inside the cell by peptidases [14]. In addition to their role in the nitrogenous nutrition of L. lactis, the peptidases can also have an important role in the development of flavors during the maturation of certain cheeses.

Ten peptidase genes have been cloned and their products characterized biochemically in L. lactis. They are grouped into different classes according to the position and nature of the peptide bond that they hydrolyze and often have a broad specificity [14]. Few studies are available at present on the regulation of the expression of these peptidases in L. lactis and solely the regulation of the wall protease has been studied in an in-depth manner [18, 19].

### SUMMARY OF THE INVENTION

This invention relates to a mutant lactic bacteria with a capacity for overexpressing one or more peptidases, wherein gene codY is inactivated.

This invention also relates to a recombinant vector for identifying or selecting mutant lactic bacteria with a capacity for overexpressing one or more peptidases, wherein gene code Y is inactivated, including a marker gene fused to a peptidase gene or a promoter of the gene, a replication origin inactivated after integration in the bacteria, an antibiotic marker and at least a part of gene cluA.

This invention further relates to a method for identifying or selecting a mutant lactic bacterium with a capacity for overexpressing one or more peptidases, wherein gene code Y is inactivated, including transferring a peptidase gene or a promoter of the gene into a bacterium by conjugation with a vector including a marker gene fused to a peptidase gene or a promoter of the gene, a replication origin inactivated after integration in the bacteria, an antibiotic marker and at least a part of gene cluA, culturing the bacteria in the presence of peptides, and measuring activity of the reporter gene.

### BRIEF DESCRIPTION OF THE DRAWINGS

Other advantages and characteristics of the invention will become manifest from the examples below. These examples concern the production by mutagenesis of mutants of L. lactis according to the invention, and with reference to the attached figures in which:

FIG. 1 shows luciferase activity of different transcriptional fusions at optical density (OD) 0.4 in amino acid (AA) chemically defined medium (CDM) and casitone (Cas) chemically defined medium.

FIG. 2 shows the repression of the transcription in A of the regulated promoters and in B of the unregulated promoters. These results were obtained from the extraction of the total mRNAs of the wild strain cultured in CDM+amino acids (AA) and CDM+casitone (Cas) at different optical densities (0.2, 0.6, 0.8, 1.2). The hybridizations were performed with different specific probes of the peptidase promoters. The regulated promoters correspond to a decrease in the RNA under growth conditions in the presence of casitone, which is the reflection of a repression of the transcription.

FIG. 3 is a schematic representation of the different factors intervening in the expression of the peptidases of L. lactis and which enabled conception of the different mutants of the invention.

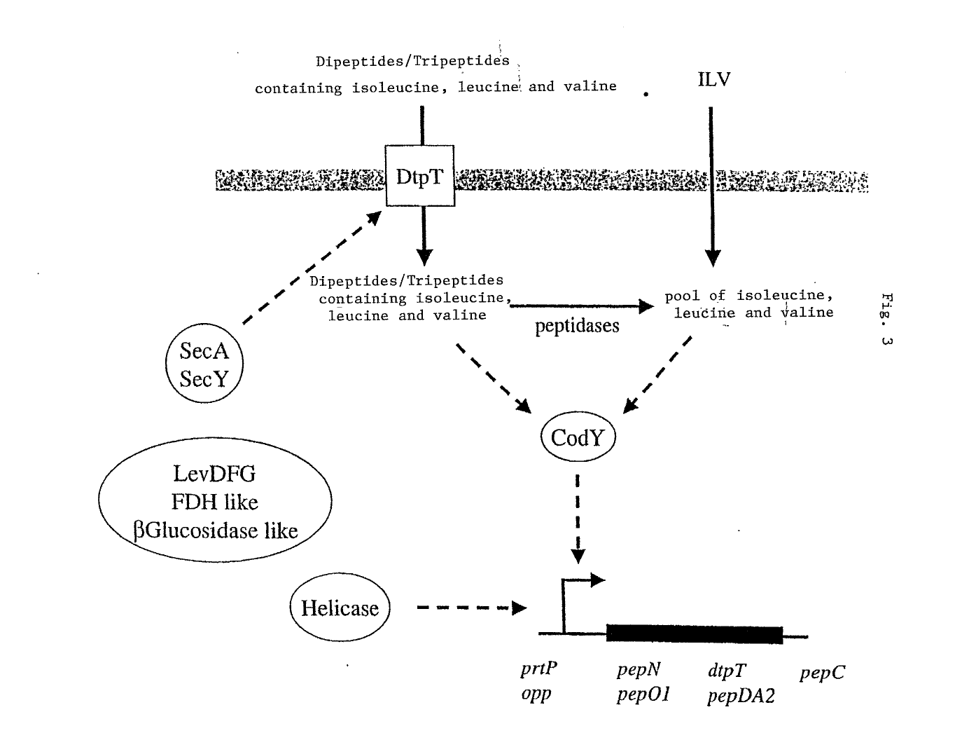

FIG. 4 shows an alignment of the sequences of the codY genes of L. lactis and of Bacillus subtilis.

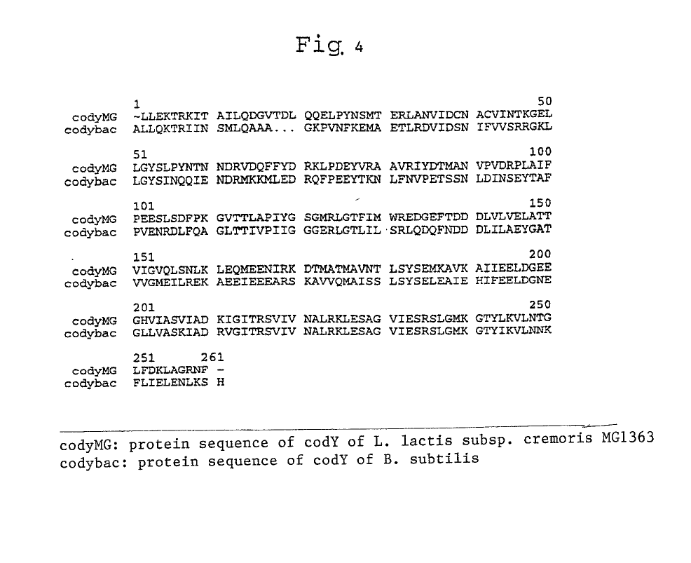

### DETAILED DESCRIPTION

Expression of certain genes in Lactococcus lactis can be critical in cheese fabrication processes. Thus, we took into consideration that evaluating the level of expression of these genes can be performed by determining the efficacy of their promoters since transcription is one of the parameters that control the expression of genes. We, therefore, developed tools based on the use of reporter genes. They constructed vectors suitable for the systematic study of numerous promoters in different cellular and environmental contexts and which can be easily transferred into a large number of strains of L. lactis. These vectors were used to study the variability of expression of the enzymes of the proteolytic system of L. lactis. The expression of sixteen genes coding for the enzymes implicated in the proteolysis of the strain L. lactis subsp. cremoris MG1363 as well as two genes of the wall protease of strains WG2 and SK11 could be characterized either by means of the vectors we developed or by detecting messenger RNAs using the Northern blot technique [8].

The studies carried out in the framework of the present invention on the characterization of the expression of the genes coding for the peptidases, proteases and transport proteins of L. lactis enabled demonstration of a coordinated regulation of their expression and, thus, determination of the factors that can affect this expression. We accordingly performed a systematic study of the transcription of the sixteen genes referred to above as implicated in proteolysis.

We thereby showed that the transcription of eight of these sixteen tested genes is regulated and repressed simultaneously by dipeptides via the intracellular pool of branched amino acids: isoleucine, leucine and valine. It is a question of the promoters of the genes of the following peptidases:

prtP, which is the wall protease [14] and, more particularly, prtPWG, which is a wall protease isolated from strain WG2, and prtPSK11, which is a wall protease isolated from strain SK11;

pepN and pep C, which are the aminopeptidases of major general specificity in the cell [14];

pepO, which is an endopeptidase implicated in the degradation of oligopeptides [14];

opp, which is the operon coding for the oligopeptide entry system [14];

dtpt, which codes for a transport protein of the hydrophilic dipeptides and tripeptides [14];

pepDA2, which codes for a general dipeptidase.

We also showed that the transcription of the genes of the proteolytic system in L. lactis is regulated by the products of diverse genes. We can cite, most particularly, the gene codY which constitutes a central regulator repressing transcription of the genes of the proteolytic system in L. lactis. We can also mention one of the genes of the operon lev and a gene coding for a β-glucosidase.

We, therefore, implemented a random mutagenesis strategy applied to the strain MG1363 [21] to find the regulators of the transcription of these peptidases. The fusion of the second promoter of the operon opp-pepO (PpepOA) to the gene of the β-galactosidase was used as a reporter to visualize the mutants whose transcription of this promoter is deregulated. We thereby isolated mutants of L. lactis obtained by insertion of a transposon.

Thus, an advantage of the invention is mutants of lactic bacteria with a capacity for overexpressing one or more peptidases, characterized in that at least one of the negative regulation factors of at least one of the peptidase genes of said bacteria is inactivated.

This inactivation can be total or partial. The term “total inactivation” is understood to mean that the factor is not expressed at all, whereas the term “partial inactivation” is understood to mean that said factor is still expressed but not sufficiently for observing the negative regulation effect found in a nonmutated bacterium.

The invention concerns most particularly mutants of lactic bacteria with a capacity for overexpressing one or more peptidases, characterized in that at least one of the negative regulation factors of at least one of the peptidase genes of the bacteria is inactivated, the negative regulation factor being selected from among the group comprising the gene codY, the genes of the operon lev, a gene coding a protein homologous with a β-glucosidase.

A first form of implementation of such an inactivation consists of a modification of the DNA sequence of one of the genes or a sequence implicated in the expression or regulation of the gene. A second form of implementation of such an inactivation consists of a modification of a gene coding for a cofactor protein required for the activity of one of the genes and/or the modification of a gene implicated in the expression or regulation of this cofactor protein.

As mutant lactic bacteria according to the invention, the mutants of L. lactis and S. Thermophilus are preferred.

The term “mutants of lactic bacteria” according to the invention is understood more specifically to refer to bacteria that have been genetically modified in a manner such that at least one of the negative regulation factors of at least one of the genes of the peptidases is totally or partially inactivated. Inactivation is understood to mean the modification of one or more genes coding for proteins constituting one or more negative regulation factors of the genes of the peptidases such as, for example, codY or the modification of one or more genes coding for the proteins required by the negative regulation factors of the peptidase genes, such as for example the transport elements of the branched amino acids or a protein required for the activity of CODY. The mutants of lactic bacteria according to the invention are advantageously obtained by mutagenesis.

The invention pertains, more particularly, to mutants of lactic bacteria with a capacity for overexpressing one or more peptidases, characterized in that at least one of the negative regulation factors common to multiple genes of the peptidases is inactivated.

The invention concerns, more particularly, mutants of lactic bacteria and especially of Lactococcus lactis at least one of whose negative transcription regulation factors common to at least two and preferably three of the promoters of the peptidase genes is inactivated.

The promoters of the peptidase genes at least one of whose negative transcription regulation factors is inactivated are, for example, selected from among the genes prtP, pepN, pepC, pepX, pepO, pepDA2, dtpT and the operon opp.

Reference will be made below to the attached sequence listing in which:

SEQ ID No. 1 represents the sequence of the gene codY of L. lactis MG 1363.

SEQ ID No. 2 represents the sequence of the gene dtpt of L. lactis MG1363.

SEQ ID No. 3 represents the sequence of the gene secA of L. lactis IL1403.

SEQ ID No. 4 represents the sequence of the gene secY of L. lactis IL403.

SEQ ID No. 5 represents the sequence of the operon lev of L. lactis IL403.

SEQ ID No. 6 represents a sequence fragment of a gene of L. lactis MG1363 whose product is homologous to a β-glucosidase.

SEQ ID No. 7 represents the sequence of the gene of L. lactis MG1363 whose product is homologous to a formate dehydrogenase.

SEQ ID No. 8 represents a part of the sequence of the gene codY of S. thermophilus.

SEQ ID No. 9 represents the complete sequence of the gene codY of S. thermophilus.

A first negative regulation factor of the peptidases of lactic bacteria, notably of L. lactis, we identified is constituted by the intracellular pool of branched amino acids which repress transcription of multiple peptidase genes. A first type of mutant is, thus, characterized by modifying this intracellular pool of branched amino acids. “Modification” is preferably understood to entail a decrease in the amount of branched amino acids. An example of modification of the pool of branched amino acids consists of selectively modifying their entry into the cell, notably by blocking one or more of the transport systems:

of the amino acids,

of the dipeptides and tripeptides,

of the oligopeptides.

The mutants of L. lactis of which at least one of the transport systems of branched amino acids, dipeptides, tripeptides or oligopeptides is blocked are mutants in which at least one of the genes coding for an element of these transport systems is inactivated.

Mutants of a dipeptide and tripeptide transport system were obtained by random mutagenesis in the gene dtpT [10] of L. lactis whose sequence is given in the attached sequence listing under SEQ ID No. 2. The studies in the prior art on this gene never revealed that there could be mutants of Lactococcus lactis with a capacity for overexpressing one or more peptidases. An example of gene dtpT modified in a mutant is characterized by inserting, for example, the plasmid pGhost-IIS1 after the nucleotides in positions 280 and 470 in sequence SEQ ID No. 2.

Repression of the transcription of the genes coding for the peptidases is raised in the mutants of the invention and, as previously stated, can result from the variation of the intracellular pool of branched amino acids. Variation of the intracellular pool of branched amino acids can also stem from variation of the degradation of the peptides (dipeptides, tripeptides or oligopeptides). Consequently, the mutants of lactic bacteria and, more particularly, of L. lactis are mutants in which at least one of the genes coding the peptidases responsible for the degradation of these dipeptides, tripeptides or oligopeptides is inactivated.

An important negative regulation factor we identified is the product of the gene codY which represses the transcription of multiple peptidases at the level of their promoter. In fact, we obtained by mutagenesis mutants of L. lactis inactivated in a gene which is homologous with the gene codY of Bacillus subtilis. In a mutant codY we reconstructed by directed mutagenesis, it was seen that transcription of the gene pepOA and transcription of at least three peptidase genes are not repressed by the dipeptides. Thus, inactivation of the gene codY in L. lactis enables augmentation of the expression of the genes of multiple peptidases by a factor of about 4 to about 55 in a medium containing a source of peptides that normally represses them in the wild strain.

The DNA sequence of the gene codY of L. lactis and the sequence of the protein codY for which it codes are represented in attached SEQ ID No. 1.

We also demonstrated the presence of the gene codY in S. thermophilus which, like L. lactis, is a lactic bacterium. The partial DNA sequence of the gene codY of S. thermophilus and the protein sequence for which it codes are represented in attached SEQ ID No. 8. The complete DNA sequence of the gene codY of S. thermophilus and the protein sequence for which it codes are represented in attached SEQ ID No. 9. Thus, the complete or partial inactivation of the gene codY in other lactic bacteria (Streptococcus, Lactobacillus, Pediococcus, Leuconostoc) can also enable augmentation of the expression of peptidases.

Consequently, a preferred type of lactic bacteria mutants according to the invention, more particularly of L. lactis, is characterized by inactivating the gene codY. A first example of such an inactivation consists in a modification of the DNA sequence of the gene codY, more particularly of the attached sequences SEQ ID No. 1, 8 or 9, or of a sequence implicated in the expression or regulation of this gene. A second example of such an inactivation consists of a modification of a gene coding for a cofactor protein required for the activity of the gene codY and/or the modification of a gene implicated in the expression or regulation of this cofactor protein.

As previously stated, in the bacteria that are mutant for codY of the invention the expression of at least three peptidases is augmented from about 4 to about 55 times in a medium containing a source of peptides which normally repress their expression. A bacterium mutant for codY according to the invention interrupts the cascade of regulation which leads to repression of the peptidases via the pool of peptides of the external medium. A change of the DNA sequence in the gene codY or in its regulation sequence consists, for example, of a mutation or a deletion which can be implemented by well known mutagenesis methods. Thus, we recorded 13 mutants of codY with, for example, insertion of the plasmid p Ghost-IIS1 after the nucleotides in positions 87, 112, 122, 289, 313, 409, 575, 604, 641, 693, 821, 877 and 882 in the sequence SEQ ID No. 1.

As previously stated, we characterized other mutants of lactic bacteria, notably of L. lactis, with a capacity for overexpressing one or more peptidases. These are mutants in which at least one of the negative transcription regulation factors of one or more genes of said peptidases is inactivated. The following can be cited as examples of such mutants:

The mutants in which a gene coding for the proteins implicated in the secretion of the transport proteins of dipeptides or tripeptides is inactivated. It is a question more particularly of mutants in which at least one of the genes secA [3] or secY [13] is modified. The proteins coded by these genes can intervene in the translocation of the protein DtpT which is implicated in the transport of the dipeptides and tripeptides. The sequences of the genes secA and secY of L. lactis are given in the attached sequence listing under numbers SEQ ID No. 3 and SEQ ID No. 4, respectively. Mutants for secA were prepared by insertion of the plasmid pGhost-IIS1 after the nucleotides in positions 1689 and 1698 in sequence SEQ ID No. 3. Mutants for secY were prepared by insertion of the plasmid pGhost-IIS1 after the nucleotides in positions 1273 and 1281 in sequence SEQ ID No. 4.

The mutants in which one of the genes of the operon lev is inactivated [17]. The genes of the operon lev code for a transport system of sugars. The sequence of the operon lev of L. lactis is given in the attached sequence listing under SEQ ID No. 5. Mutants for the operon lev according to the invention were prepared by insertion of the plasmid pGhost-IIS1 after the nucleotides in positions 40, 108, 1075, 1140, 1145 and 2735 in sequence SEQ ID No. 5.

The mutants in which at least one of the genes having homology with a gene coding a protein whose structure is of the type of that of a β-glucosidase and/or a formate dehydrogenase is inactivated. The sequence of a gene coding this protein homologous with a β-glucosidase is given in the attached sequence listing under number SEQ ID No. 6. The sequence of a gene coding a formate dehydrogenase is given in the attached sequence listing under number SEQ ID No. 7.

As above, the term an “inactivated gene” is understood to mean a gene whose sequence or a sequence implicated in its expression or its regulation is modified.

Consequently, another preferred type of mutant of lactic bacteria according to the invention, more particularly of L. lactis, is characterized by the inactivation of one of the genes of the operon lev. A first example of such an inactivation consists of a modification of the DNA sequence of one of the genes of the operon lev, more particularly, of the attached SEQ ID No. 5, or a sequence implicated in its expression or the regulation of one of the genes of this operon. A second example of such an inactivation consists of a modification of a gene coding for a cofactor protein required for the activity of one of the genes of the operon lev and/or the modification of a gene implicated in the expression or regulation of this cofactor protein.

A third preferred type of mutant of lactic bacteria according to the invention, more particularly, of L. lactis is characterized by the inactivation of a gene coding for a protein homologous with a β-glucosidase. A first example of such an inactivation consists of modification of the DNA sequence of a gene coding for a β-glucosidase, more particularly, of attached SEQ ID No. 6, or of a sequence implicated in the expression or regulation of this gene. A second example of such an inactivation consists of a modification of a gene coding for a cofactor protein required for the activity of a gene coding for a β-glucosidase and/or the modification of a gene implicated in the expression or regulation of this cofactor protein.

It is understood that the mutants according to the invention can also be characterized by more than one of the mutations described above.

As previously stated, we developed tools based on the use of reporter genes such as the luciferase gene of Vibrio harveyi. The expression of luciferase, which is detected by an emission of light, makes it easy to measure the activity of promoters, even in complex media [4]. The vectors pVar we constructed contain a replication origin inactivated after integration, an antibiotic marker and a part of the gene cluA [6]. This latter fragment allows the plasmid to integrate itself by homologous recombination in the sex factor. This factor is a conjugative element of 60 kb present in integrated form in the chromosome of certain strains of L. lactis. The integrated constructions in the sex factor at the level of the gene cluA in a strain can thus be transferred into numerous strains of L. lactis by conjugation.

The invention, thus, also concerns a recombinant vector for identifying or selecting mutant bacteria according to the invention. This vector is characterized in that it comprises a marker gene fused to a peptidase gene or a promoter of this gene, a replication origin inactivated after integration in the bacterium, an antibiotic marker and a part of the gene cluA.

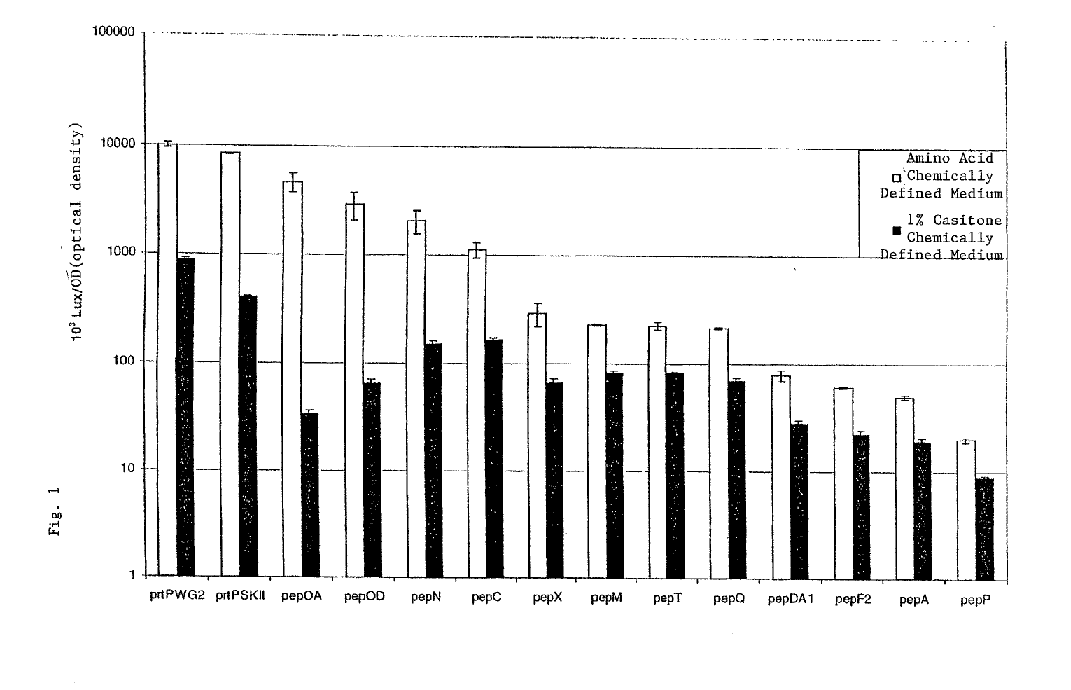

Such a vector makes it possible to distinguish a mutant strain according to the invention from a wild strain incapable of overexpressing one or more peptidases. The following method can be used to distinguish the strains. A vector pVar containing the promoter PpepOA of the operon opp-pepO, fused to the luciferase gene, is transferred into the strains by conjugation to determine the level of expression of peptidases in the strains. Measurements of the luciferase activity under the control of PpepOA indicate whether the transcription of at least the gene pepO is derepressed in these strains. Constructions with other promoters enable verification of the number of peptidase genes whose transcription is derepressed. The reference luciferase activities of the wild strain which reflect the repression of the transcription of the peptidase genes during growth in the presence of peptides, via the pool of branched amino acids, are listed in FIG. 1.

Consequently, the invention also concerns a method for the identification or selection of a mutant lactic bacterium according to the invention, characterized in that a peptidase gene or a promoter of this gene is transferred into a bacterium by conjugation with the vector defined above, that the bacterium is cultured in the presence of peptides and, using any appropriate means, the activity of the reporter gene is measured which reflects the repression of the transcription of the peptidase genes.

The reporter gene is advantageously the luciferase gene.

The invention also concerns the use of mutants of lactic bacteria as described above or a mixture of them in a cheese fabrication and/or maturation process. In an advantageous manner, the mutants of lactic bacteria as described above or a mixture of them are used in a fabrication and/or maturation process for soft or pressed cheeses.

I—Material and Methods

1) Bacteria Strains, Media, Vectors and DNA Manipulations

Strain L. lactis MG1363 was cultured at 30° C. in M17 glucose medium. If necessary, 5 μg/ml of erythromycin was added to the culture medium. The peptidase promoters under study (PepP, PepA, PepF2, PepDA1, PepOA, PepQ, PepX, PepOD, PepM, PepT, PepN and PepC) and the two promoters of the operon opp (pepOA/pepOD) were amplified by PCR by means of specific primers from the chromosome of the strain L. lactis subsp. cremoris MG1363. A series of conditional replication vectors containing luciferase reporter genes of Vibrio harveyi and a fragment of the sex factor (gene cluA) was constructed. pVar-1 was used for fusing to the luciferase genes the DNA fragments obtained by PCR and corresponding to the different promoters.

2) Integration of the Transcriptional Fusions on the Chromosome of MG1363 and Conjugation

After transformation of strain MG1363 by the plasmids pVar-1 containing the peptidase promoters, the fusions were integrated in the chromosome by homologous recombination, either at the peptidase promoter locus or at the locus of the sex factor (in the gene cluA). Identification of the integration locus was performed by means of suitable primers by PCR amplification and hybridization of a Southern gel. Transfer of the sex factor was implemented by conjugation between two strains [5].

3) Determination of the Luciferase Activity in L. lactis

Measurements of the luciferase activity were performed on the Berthold Lumat luminometer LB9501. A milliliter of L. lactis culture was mixed with 5 μl of nonaldehyde and the light emission directly measured. The peak value was brought to OD_{600 nm} of the culture and luciferase activity was measured during the entire growth. The luciferase activity shown in FIG. 1 was measured at OD_{500 nm}=0.4 and expressed in 10³ lux/OD.

4) Chemically Defined Medium (CDM)

This chemically defined medium (CDM) is defined in Sissler et al. [22]. The nitrogen source of this medium is a mixture of amino acids. In the “casitone CDM”, there was added an extract of casitones (milk caseins degraded by pancreatic enzymes) which is a source of small peptides.

5) Constructions: PpepOA-βgal.

The second promoter of the operon opp-pepO (PpepOA) of strain MG1363 was amplified by the following oligonucleotides (GGGAATTCTTTGGGAACAATGATAA and CGGGATCCGTTACTTCTGAACCA) and the amplified 500-pb fragment was cloned in the plasmid pJIM762 at the site EcoRI-BamHI upstream of the β-galactosidase gene of Escherichia coli (E. Guédon, unpublished results). This plasmid, whose β-galactosidase gene is under the control of the expression signals of PpepOA, was integrated into the chromosome of MG1363 by homologous recombination at the promoter locus. The transcription at PpepOA was repressed by the dipeptides contained in the casitone of the medium and the strain containing the fusion was white on a chemically defined medium (CDM) containing casitones. Transcription at PpepOA was derepressed on a CDM containing amino acids as nitrogen source and the strain containing the fusion was blue (the strain was cultured with phospho-β-galactoside (P-β-gal) in both cases).

6) Plasmid Pghost8-ISS1

This conditional replication plasmid (heat-sensitive replication protein) had a tetracycline antibiotic marker and an insertion sequence ISS1 [16]. Augmentation of the temperature from 30° C. to 37° C. inhibited replication of this plasmid and the tetracycline-resistant strains obtained contained the plasmid integrated in the chromosome. It integrated itself in a random manner in the chromosome of L. lactis by replication transposition [16].

7) Mutagenesis By Transposition

Random mutagenesis was performed with the heat-sensitive plasmid pGhost8-ISS1 in strain MG1363 containing the fusion promoter PpepOA-β-gal. In casitone CDM, in the presence of P-β-gal, out of 50,000 white clones isolated, 46 had a phenotype of blue color. Expression of β-gal fusion was derepressed in these mutants. The plasmid pGhost8-ISS1 was, thus, inserted in a gene whose product is a direct or indirect repressor of the expression of PpepOA.

8) Identification of the Mutants By Cloning Junctions

Transposition by ISS1 into the chromosome yields an insertion of pGhost8 surrounded by a duplicate copy of ISS1. The junctions were cloned using unique restriction sites (EcoRI and HindIII) present on pGhost8. Digestion of the chromosome by these enzymes made it possible to obtain the plasmid pGhost8 containing the flanking regions. The transposition site was thereby characterized by sequencing the junctions with the following oligonucleotides (for the junction EcoRI: TCACCTCATATAAATTCCCCA and AAATGGAACGCTCTTCGG) (for the junction HindIII: CGCCAGGGTTTTCCCA GTCACGAC and ACCAACAGCGACAATAATCACA).

9) Mutant codY

Random mutagenesis made it possible to obtain, among others, mutant codY for which the transcription of multiple genes coding for the proteolytic enzymes is deregulated. Inactivation of this gene in L. lactis augments expression of the genes opp-pepO, pepN and pepC by a factor of 55, 14 and 4, respectively, in CDM with casitones in which the expression was normally repressed by the peptide source.

10) Inactivation of codY By Simple Crossing-Over

A 540-pb PCR fragment was amplified by the following oligonucleotides (CAGTATGACTGAACGCTTGGC and GCGATAACATGCCCTTCTTCA) and cloned in the plasmid pJIM2242. This plasmid was integrated into the gene codY by simple crossing-over and a mutant codY was verified by Southern hybridization. This mutant has the same phenotype as the codY mutants obtained by mutagenesis.

11) Other Mutants

Random mutagenesis enabled identification of many other codY mutants. Mutations of the genes dtpT, of the operon lev, secA, secY, and genes coding for a helicase, a β-glucosidase and an enzyme homologous with a formate dehydrogenase also led to mutants overexpressing at least pepO or additional peptidases.

II—Results

1) Construction of the Vector pVar-1

Integrative vectors making it possible to follow the expression of a reporter gene under the control of a promoter on the chromosome were constructed. The luciferase genes of Vibrio harveyi were used as a reporter gene. The luciferase activity of the transcriptional fusions with the promoters of peptidase genes is the reflection of the expression of the peptidase genes [11, 20]. A vector which replicates itself in a conditional manner was used to integrate the transcriptional fusions on the chromosome [15]. This vector was conceived to be easily transferable by conjugation, in particular, in industrial strains that are difficult to transform. A fragment (gene cluA) of the 60-kb chromosome element called the “sex factor”, which is possessed by certain strains of lactococci, was introduced into this vector. This element is capable of high-frequency self transfer in the species L. lactis [7]. By integrating our transcriptional fusions into this sex factor, these fusions were rendered transferable to other strains of lactococci by conjugation of the sex factor. Among different vectors constructed, the pVar-l contains an erythromycin-resistance gene in addition to the components described above. It was verified that the transcriptional fusions integrated at the promoter locus or in the gene cluA with pVar-1 had identical luciferase activities [9].

2) Expression of Fusions with the Promoters of Genes Coding for Peptidases

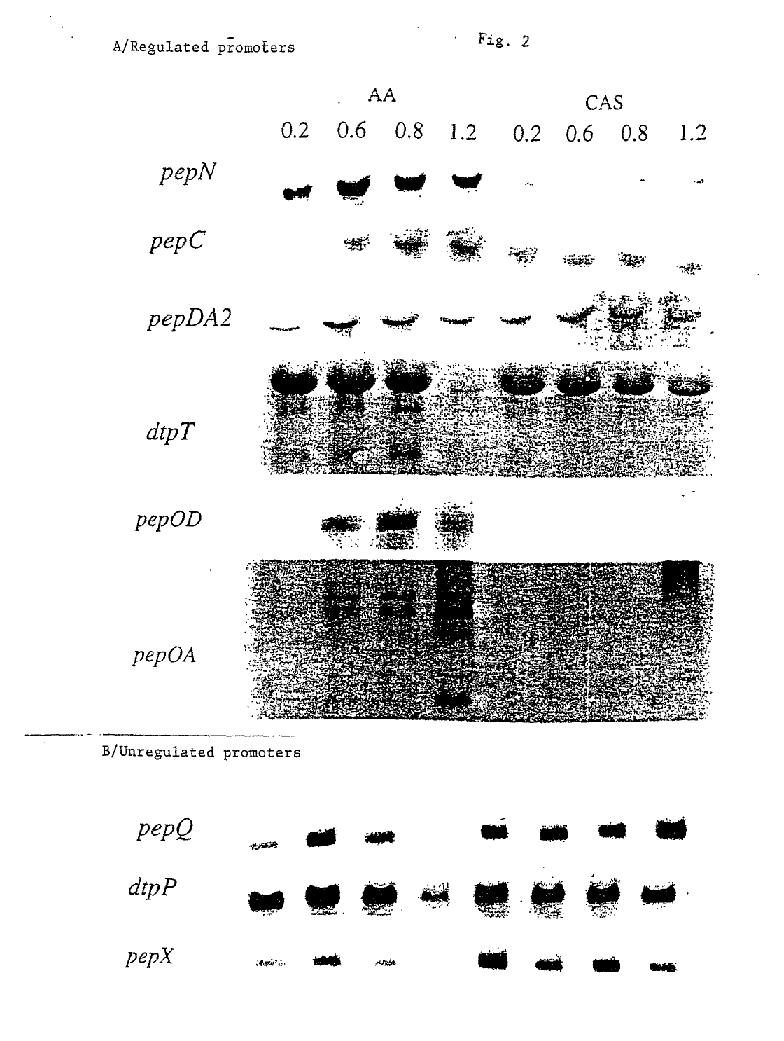

The promoters of 11 genes coding for peptidases (pep) of L. lactis MG1363: pepA, pepC, pepDA1, pepF2, pepM, pepN, pepP, pepQ, pepT, pepx; and the two promoters of the operon opp-pepO (PpepOA and PpepOD) in which is found the gene pepO, were cloned, fused to the gene lux in the vector pVar-1 and integrated by homologous recombination into the chromosome of L. lactis MG1363 at the locus of the different promoters. Both protease promoters (prt of strains WG2 and SK11) were fused to the luciferase gene on a plasmid. Expression of these fusions was determined in CDM and casitone CDM, and the values of the luciferase measurements are presented in FIG. 1. In CDM, the fusions were grouped together into different classes on the basis of the measured luciferase levels. The strongest luciferase activity was obtained with the plasmid fusions containing the promoters of the genes prt (10·10³ lux/OD (10³)). For the chromosome fusions, the strongest luciferase activity was obtained with the promoters PpepN, PpepC, PpepOA and PpepOD (1 to 5 10·10³ lux/OD (10³), an average activity was obtained with the promoters of the genes pepQ, pepX, pepM and pepT (200 to 300 lux/OD (10³)) and weak activity was obtained with the promoters of the genes pepP, pepA, pepF2 and pepDA1 (20 to 80 lux/OD (10³)). It should be noted that the highest expression levels were obtained with the fusions containing the promoters of the genes coding for the peptidases of very broad specificity (pepC, pepN, pepO) and for a transport system of oligopeptides (Opp) which is essential for the growth of lactococci in a milk medium. Expression of peptidase genes was diminished in casitone CDM medium which contains a nitrogen source constituted by amino acids and peptides (FIG. 1). The force of the promoters PpepP, PpepA, PpepF2, PpepDA1, PpepQ, PpepT and PpepM was diminished 2 to 3 times in casitone CDM, whereas the force of the promoters PpepX, PpepC, PpepN, PprtPWG2, PprtPSK11, pepO and the operon opp-pepO was repressed 5, 7, 13, 21, 12 and 153 times, respectively, by the dipeptides of the culture medium via the pool of branched amino acids in the cell. Northern Blot analysis of the transcripts confirmed the results obtained with the transcriptional fusions and revealed that the transcription of the genes pepDA2 and dtpT but not that of dtpP was repressed by the peptides of the casitone (FIG. 2).

3) Obtaining and Characterizing Derepressed Mutants

On a peptide-rich medium and in the presence of P-βgal, the wild strain containing the fusion PpepOA-βgal yielded white colonies because expression of the fusion is repressed. The mutants obtained yield blue colonies because expression of the fusion PpepOA-βgal is derepressed.

Different genes of the mutants in which pGhost was inserted were identified (codY, dtpT, the operon lev, secA, secY and the genes coding for a β-glucosidase and an enzyme homologous with a formate dehydrogenase (fdh)). The Northern Blot analysis of the mRNAs of a mutant strain cultured in a peptide-rich medium confirmed that the transcription of the gene pepO was no longer repressed in the mutants of the genes codY, dtpT, fdh and the operon lev. The derepression of pepO in the mutants of the genes coding for secA, SecY and β-glucosidase has yet to be confirmed.

4) Characterization of the Expression of Peptidases in the Deregulated Mutants

Transcription of the peptidase genes was characterized in the mutants by measurement of activity. Two classes of mutants could be obtained: one class for which the transcription of multiple peptidases is derepressed (pleiotropic mutants) and the other class in which only the transcription of the gene pepO is derepressed.

Mutants of the genes codY, dtpT: in an M17 rich medium containing repressor peptides, the luciferase activities were measured in a wild strain and in a codY mutant for the promoters PpepOD, PpepC and PpepN, and in a dtpT mutant for the promoter PpepOD. In a mutant codY, repression of the transcription diminished by a factor of 35, 4 and 14, respectively for the genes pepOD, pepC and PepN. In a mutant dtpT, repression of the transcription diminished by a factor of 15 for the gene pepOD.

### BIBLIOGRAPHIC REFERENCES

1) Boutrou R., Sepulchre A., Gripon J. C., Monnet V., 1998. Simple test for predicting the lytic behavior and proteolytic activity of lactococcal strains in cheese. J. Dairy Sci., 81, 2321-2328.

2) Delorme C., Ehrlich S. D., Renault P., 1999. Regulation of expression of the Lactococcus lactis histidine operon. J. Bacteriol., 181, 2026-2037.

3) Driessen A., Fekkes P., van der Wolk J. P., “The Sec system” 1998, Curr. Opin. Microbiol., 1(2), 216-222.

4) Drouault S., Corthier G., Delorme C., Ehrlich S. D., Renault P., 1998. Metabolic regulations of Lactococcus lactis in pure or mixed culture in milk. Lait, 77, 15-23.

5) Gasson M. J., Swindell S., Maeda S., Dodd H. M., 1992. Molecular rearrangement of lactose plasmid DNA associated with high-frequency transfer and cell aggregation in Lactococcus lactis 712. Mol. Microbiol., 6, 3213-3223.

6) Godon J. J., Jury K., Shearman C. A., Gasson M. J., 1994. The Lactococcus lactis sex-factor aggregation gene cluA. Mol. Microbiol., 12, 655-663.

7) Godon J. J., Pillidge C. J., Jury K., Gasson M. J., 1996. Characterization of an original conjugative element, the sex factor of Lactococcus lactis 712. Lait, 76, 41-50.

8) Guédon E., Renault P., Ehrlich S. D., Delorme C. “Environmental factors involved in the transcriptional regulation of 18 proteolysis components in Lactococci” (submitted for publication).

9) E. Guédon, P. Renault, S. D. Ehrlich and C. Delorme. “Evaluation of the diversity of genetic expression in lactococci: development of a tool and its application to peptidases.” (Accepted for publication in “Science des aliments”).

10) Hagting A. et al., 1994, “The di- and tri-peptide transport protein of Lactococcus lactis”, J. Biol. Chem., 269, 11391-11399.

11) Hill, P. J., Rees, C. E. D., Winson, M. K., Stewart, G. S. A. B., 1993. The application of lux genes. Biotechnol. Appl. Biochem., 17, 3-14.

12) Juillard V., LeBars D., Kunji E. R., Konings, W. N., Gripon, J. C., Richard, J., 1995. Oligopeptides are the main source of nitrogen for Lactococcus lactis during growth in milk. Appl. Environ. Microbiol., 61, 3024-3030.

13) Koivula T., Palva I., Hemila H., “Nucleotide sequence of the secY gene from Lactococcus lactis and identification of conserved regions by comparison of four secY proteins”, 1991, FEBS Lett. 288:114-8.

14) Kunji E. R., Mierau I., Hagting A., Poolman B., Konings, W. N., 1996. The proteolytic systems of lactic acid bacteria. Antonie Van Leeuwenhoek, 70, 187-221.

15) Law J., Buist G., Haandrikman A., Kok J., Venema G., Leenhouts K., 1995. A system to generate chromosomal mutations in Lactococcus lactis which allows fast analysis of targeted genes. J. Bacteriol., 177, 7011-7018.

16) Maguin E., Prevost, Ehrlich S. D., Gruss A., “Efficient insertional mutagenesis in lactococci and other Gram-positive bacteria”, 1996, J. Bact. 178, 931-935.

17) Martin-Verstraete I., Debarbouille M., Klier A., Rapoport G. “Levanase operon of Bacillus subtilis includes a fructose specific phosphotransferase system regulating the expression of the operon” 1990, J. Mol. Biol., 244, 657-671.

18) Marugg J. D., Meijer W., Van Kranenburg R., Laverman P., Bruinenberg P. G., DeVos W. M., 1995. Medium-dependent regulation of proteinase gene expression in Lactococcus lactis, control of transcription initiation by specific dipeptides. J. Bacteriol., 177, 2982-2989.

19) Meijer W., Marugg J. D., Hugenholtz J., 1996. Regulation of proteolytic enzyme activity in Lactococcus lactis. Appl. Environ. Microbiol., 62, 156-161.

20) Renault P., Corthier G., Goupil N., Delorme C., Ehrlich S. D., 1996. Plasmid vectors for gram-positive bacteria switching from high to low copy number. Gene. 183, 175-182.

21) Prevost H., Ehrlich S. D., GrussA., 1996, Efficient insertional mutagenesis in lactococci and other gram-positive bacteria. J. Bacteriorl., 178:931-935.

22) Sissler M., Delorme C., Bond J., Ehrlich S. D., Renault P., Francklyn C., 1999, “An aminoacyl-tRNA synthetase paralog with a catalytic role in histidine biosynthesis” PNAS, 96:8985-8990.

1. A mutant lactic bacteria with a capacity for overexpressing one or more peptidases, wherein gene codY is inactivated.

2. The mutant lactic bacteria according to claim 1, wherein said inactivation is total or partial.

3. The mutant lactic bacteria according to claim 1, wherein the DNA sequence of said gene or of a sequence implicated in expression or regulation of said gene is modified.

4. The mutant lactic bacteria according to claim 1, wherein a gene coding for a cofactor protein required for activity of said gene and/or modification of a gene implicated in expression or regulation of this cofactor protein is modified.

5. The mutant lactic bacteria according to claim 1, wherein the lactic bacterium is L. lactis.

6. The mutant lactic bacteria according to claim 1, wherein the lactic bacterium is S. thermophilus.

7. A recombinant vector for identifying or selecting mutant lactic bacteria according to claim 1, comprising a marker gene fused to a peptidase gene or a promoter of said gene, a replication origin inactivated after integration in the bacteria, an antibiotic marker and at least a part of gene cluA.

8. A method for identifying or selecting a mutant lactic bacterium according to claim 1, comprising:
transferring a peptidase gene or a promoter of said gene into a bacterium by conjugation with a vector comprising a marker gene fused to a peptidase gene or a promoter of said gene, a replication origin inactivated after integration in the bacteria, an antibiotic marker and at least a part of gene cluA;
culturing said bacteria in the presence of peptides; and
measuring activity of the reporter gene.


SEQUENCE LISTING (29 sequences)

SEQ ID NO: 1 - DNA, 937 bp, Lactococcus lactis
  CDS (121)..(897): sequence of codY from L. lactis MG1363
agagtaatttttctgacaattttttattgtttttccatatgcttttttatgttatactgattatgaaaaattttgtataaaaacaagaatataaaaaaataggagaacaaagtggctacattacttgaaaaaacacgtaaaatcaccgcgattttgcaagatggagtgaccgatttgcaacaagagttgccatacaacagtatgactgaacgcttggcaaacgtcattgattgcaacgcctgcgtgattaatacgaagggcgagttgcttggttactcattgccttacaatacaaacaatgatcgcgttgaccaatttttctacgatcgtaaattgcctgacgaatacgttcgtgcagcagtacgtatttacgatacaatggcaaacgttcctgttgatcgtcctttagcaattttcccagaagaaagtcttagcgattttccaaaaggtgtaacaactttagcgcctatctatggttctggaatgcgtcttggaacatttattatgtggcgtgaagatggtgaatttacagatgacgatcttgttttggttgagcttgcaacaacagtaatcggtgtacaactctcaaaccttaaacttgaacaaatggaagaaaatatccgtaaagacactatggcaacaatggctgttaatacactttcttactcagaaatgaaagctgtcaaagcaattattgaagaacttgatggtgaagaagggcatgttattgcctctgtcattgctgacaagattggtattacacgttcagtgattgttaatgctttacgtaaacttgaatctgctggtgttattgaatcacgttcacttggtatgaaaggaacttatcttaaagttcttaatactggtttgtttgataaacttgctggacgtaatttctaaaagtcagagcttaacgcttgttcttttatcatttgtt

SEQ ID NO: 2 - DNA, 1389 bp, Lactococcus lactis
  CDS (1)..(1389): sequence of dtpT from L. lactis IL1403
atgcgtgccattctggtttattacctctatgcattgacaactgcagataacgcaggtttaggacttcctaaagctcaggcaatggcgattgtaagtatttatggtgcacttgtctatctttcaacaattgttgggggatgggttgctgaccggttgttgggcgcttcgcgcacaatcttcttgggtggtattttaatcactttaggacacgtcgctttagcaacaccatttggtttatcttcactcttcgtggcattattcttgattatcttaggaacagggatgcttaaacccaatatttctaacatggttgggcatctatattcaaaagatgactcacgtcgtgatactggatttaatatctttgtagtcggaattaatatgggttctctgattgctccattgattgttgggacagttggacaaggcgtgaactaccacttaggtttctcacttgccgcaatcggaatgatttttgcattatttgcttattggtatggacgtcttcgtcatttcccagaaattggacgtgaaccatctaatccaatggatgcaaaagcaaaacgtaattttattattacattaacgattgttcttatcgttgctttaatcggatttttcttaatttatcaagcaagtcctgcgaatttcatcaataatttcattaacgttttatcaattatcggtattgttgttccaattatttatttcgtaatgatgtttacctctaaaaaggtagaatcagacgaacgtcgtaaattaacggcttatattcctttgttcctttctgctattgtcttttgggcaattgaagaacaaagttctacgattattgcggtttggggagaatcacgttctaacttaaatcctacttggtttggatttactttccatattgacccatcttggtaccaattgttgaacccactcttcatcgttctcttgtcacctatctttgtacgaatttggaacaaattaggagatcgtcaaccatcaaccatcgttaaatttggtcttggactgatgttgaccggagcttcttatttgattatgacacttcctggactcttgaatgggacttctggacgtgcgagtgctctttggctagtattgatgtttgctgttcaaatggcaggtgaattacttgtttcaccagttggtttatcagtttcaacaaaattagcgccagtagcattccaatctcaaatgatggcaatgtggttcttggcagactcaacttcacaagcgattaatgcccaaattacacctatctttaaagcagcaacagaagttcacttctttgcaattacagggattatcggtattatcgttggaatcatcctccttattatcaaaaaacctattttgaaattaatgggagatgttcgt

SEQ ID NO: 3 - DNA, 1719 bp, Lactococcus lactis
  CDS (1)..(1719): sequence of secA from L. lactis IL1403
atggaaaatgttgccttaactcactttgtagataatgctttacgtgccaactttatcatgcttcacgacatcgactatatggttgatgaaaaccaagaagttttgattattgaccaatttactggacgtacgatgcctggacgtcgctattctgatggtcttcaccaagcaattgaagctaaagaagctgtgccaattcaagatgaatcaaaaacaatggcttcaattacgattcaaaactacttccggatgtataaaaaactgtcagggatgacagggactgctaaaaccgaagaagaagaattccgtgagatttataacattcaaatcacaccaattccaaccaaccgtcctgttcaacgtttagatcatccagatttactttacccaactttggaagctaaatttaaagcagttattgatgatattaaacgtcgtcatgctgaaggtcaaccaatattgattggtactgttgctgtcgaaacttccgaattgatttctaagaaattggttgaagcaaaaattcctcacgaagttttgaatgcgaaaaatcacttccgtgaagcacaaatcatcatgaatgcgggtcaacaaggagcagtaacgattgcgaccaacatggccggtcgtgggactgatatcaagcttgggcctggtgtaattgatcatgtagaccctgaattccgaggtcttgctgttattggtactgagcgtcatgaatctcgtcgtattgataatcaattacgtggtcgttctggacgtcaaggtgacccaggggtttcacaattctatctttctcttgaagatgaattaatgaaacgttttggttcggaacgtgtttcagctttcctagatagaatgcgtatttctggtgaagatgctgtcatcaaatctggcttgattactcgtcagattgaaagttcacaaaaacgtgtcgaaggaaataactacgattctcgtaaacaagtcttgcaatatgatgatgtcatccgtgagcaacgtgaagttatttatgcgcaacgtcaggaagttatcttggctacagaagatatgactcctgttttgatgggcatgttcaagcgaacaattgatcgtcaagtggatggtcatgaacttgcaggaagtcttaaagatgaagaaaatgtcaaaaatctcttgcaaacattacacaatacaatgttgccagaagatggcattgaattgtctgaactgacaggtttgtcagtacaagcaatgaaagatttgatttttgataaagtcaaagctcgttatgcttcacaaatggaaaaattatctgacccagaacgtcagttggaattccaacgtgcagttatcttacgagttgttgataataactggtcagaacacattgatgcgcttgaccaaatgcgtcaatcagtaggacttcgtggttatgcccaaaataaccctattgttgaatatcaagaagaatcatataaaatgtacaataatatgattggtgcgattgaatttgaagtgactcgtttgatgatgaaagctcaaattcaaccacaaacggcaatccgtcaggaagcgccaagaatgacaaccacagcttcacaagaaaatattacaaatgttgatactgaacattctgtcagtgaagaaatttcatttgaaaatgttggtcgtaacgacctttgtccttgtggttctggtaagaagtttaaaaattgtcacggacgtacacatattgcc

SEQ ID NO: 4 - DNA, 1317 bp, Lactococcus lactis
  CDS (1)..(1317): sequence of secY from L. lactis IL1403
atgttttttaagacgcttaaggaagcctttaaggtcaaagacgtccgagcaagaattctctttacgattttcatcctttttgttttccgcttaggtgctcatattacggtacctggcgtcaacgttcaaaacttaacagaagtaagtaatcttcctttcttgaacatgatgaacttggtttctggtaatgccatgcaaaactactcactctttgcaatgggagtttcgccttatatcactgcttcaatcattgttcaattgttgcaaatggatattttaccaaaatttgttgagtggtcaaaacaaggggaaattggacgtcgtaaactgaatcaagcgacacgttacattaccttagtgcttgctatggcacaatctatcgggattactgctggtttccaagccatgagctcgttaaatattgtgcaaaatccaaattggcaaagctatttgatgattggtgcaattttgaccactggttcaatggttgtcacttggatgggtgaacaaattaatgaccaaggttttggctcaggtgtttcagtaatcatctttgctgggattgtctctagtattccatcagccatcaaatctgtttatgatgaaaaattcttaaacgtaagaccatctgaaattcctatgtcttggatatttgttattggattgattttgtcagcaattgtcattatttatgttacaacatttgttcaacaagcggaacgtaaagtaccaattcaatacactaagttgactcaaggcgcaccaacaagttcgtactttccacttcgtgtcaatccagctggtgttatcccagttatctttgctggttcaattacaactgctcctgctacgatcttgcaattcttgcaacgttcacaaggtagcaatgtaggttggttatcaaccttacaaaacgccttgtcatatacgacttggacaggaatgctcttctacgcattattgattgttctctttactttcttctactcattcgttcaggtcaatcctgaaaagatggctgaaaaccttcaaaaacaaggctcttacattccatctgttcgtccgggtaaaggaaccgaaaagtatgtttctcgtctcttaatgcgtcttgcaacggttggttcgctcttccttggattgatttcaatcattccaattgcggcccaaaacgtttggggacttccaaaaatcgtcgctcttggagggacatcattattaatcttgattcaagttgcgattcaagcagttaaacaacttgaaggatatttacttaaacgtaaatatgcaggatttatggataatccacttgaaacaaaa

SEQ ID NO: 5 - DNA, 2916 bp, Lactococcus lactis
  CDS (127)..(1083)
  CDS (1165)..(1974)
  CDS (1996)..(2916)
  misc_feature (1)..(2916): sequence of the lev operon from L. lactis 1403
gtaatatttttatgaaaacatttgcaaatatcgatttgaagtagtataataactaagtaataatttttattataatcttatataggaggttactcacattgagtatcggaattgttattgcgagccatggtgaattcgccgcagatcaaacaatctggttctatgattttcggagagcaagaaaaagtacaagttgttacttttatgcctagcgaaggaccaactgatttgcatgctaaaatcgaagctgccatcgcaacatttgatgctgaagatgaagtacttgtccttgctgacttatggagcggttctccatttaatcaagcaagtgcagtgatgggtgaaaatccagagcgcaagattgctatcatcacaggcctcaacctgcctatgcttatccaagcctacacagaacgcatgatggatgcgtctgccggggtggataaagtcgtagcaaatattatgaaagaagccaaaggcggtattaaagtactacctgaagaacttcaacctgctgaagaaactgctgttgcagctgctccggctgctgttcaaggtgcgattcctgaaggaacagtcatcggtgatggtaaaattaaaattaacctcgctcgtattgactcacgtttgcttcacggacaagttgcaactgcttggactccagactcaagagcaaaccgcatcatcgttgtttctgacaccgtttctaaagatgaacttcgtaagaagctcattgaacaagcggctccaactggtgtaaaagctaacgttataccaattaagaaaatgattgaagttgctaaagacccacgttttggtgacactaaagcccttcttcttttcgaaacgccacaagacgctcttgcaacaatcgaaggtggcgtaccaattgaaacattgaacgttggttctatggctcactcaactggtaaaacaatgctcaacaaagttctttctatggacaaagatgacgttgctacttttgaaaaattgcgtgacctcggagttaaattcgacgtacgtaaagttccagctgactctaaatctgacctctttggtttgattaacaaagctgacgtacaataatcagaatatgctcgtatgatattctgattactaaaattgaatattaggcagccaaataattaaggagatataaaaaacatggaatacggtgttttatctgtaatcttggtcattgttgttgccttccttgctggtcttgaaggtatccttgaccaatggcaattccaccaaccaattatcgcgtgctcgctcatcggtattgttaccggtcatgcttctgcagggattatcctcggtggttcacttcaattgatcgctcttggttgggctaacgttggtgccgctgtcgcacccgatgctgcccttgcctctatcgcatcatctatcttgatggttcaatcaaataactttgacttgactcacatcatgggtactatcgttcctgctgctatcttgcttgcaactgctggtcttgtattgactactcttgtacgtatgctttcagttgtgctcgttcaccaagctgaccgtgctgctgaaaatggttcatactcaggtgttgaaatgtggcacttcatcgcgcttatctgtcaaggtttgcgtattgctatccctgctggacttcttttggttatctcaccagatgctatccaaaaagcacttgctgctattcctccagttatctctggcggtcttgctgtcggtggtgggatggttgttgccgttggttatgcaatggttatcaaccttatggctactcgtgaagtatggccattcttcttccttggtttcgctctcgcaccaatctctgaattaacattgattgcaactggtgtcctcggtgttgttatcgctatcgtttaccttaacctccaagcttctggtggttctggaaatggtactgcatcttcatcaggtgacccaattggcgacatcttgaacgactactaagaaaggaggatctaaaaaatgtctgaaaataaagtaactcttgataagaaaatccgtcgtagcgttatgtggcgttcaatgttcctccaaggttcttggaactacgaacgtatgcaaaatggtggttgggcttactcgctcattccagcattgaaaaaactctacccttctggcgaagaagctaaagaagctttgaaacgtcacttggaattctttaatactcacccatacgttgccgctcctatcatcggtgtaactcttgcccttgaagaagaacgtgctaacggtgctgatatcgatgatgccgctattcaaggggttaaagttggtatgatgggtcctcttgccggtatcggtgaccctgtcttctggtttacagtacgtcctatcgttggtgcgattgcagcttcattggctactggtggatcaattatcgctccactcttcttcttcatcgtgtggaacgctatccgtatcgctttcttgtggtacactcaagaatttggttataaatcaggttctgcaatcactaaagaccttggtggaggacttctccaaactgttactaaaggtgcatctatccttggtatgttcgtccttggtgtattgattcaacgttgggtaacaattaactttaatggtcctaacgctgttgtttcaaaaattcctttacaaaaaggtgcttatctagaattccctaaaggttctgtatctggtacacaacttcatgatattcttggtcaagttggtaacaaactttctcttgatcctacaaaagtaacttaccttcaagataacttgaatcaattgattcctggtcttgctggtttgcttatcacattcctttgcatgtggttgcttaagaaaaaagtttctccaatcgttattatctttggtctcttcgtcgtgggtatcctcggtcgatgggctcaaatcatg

SEQ ID NO: 6 - DNA, 367 bp, Lactococcus lactis
  misc_feature (1)..(367): L. lactis MG1363 gene fragment whose product is homologous to a beta-glucosidase
gactttattatctttcaaaagttgataggtgtttttatttcatctgttaaaattattgtttacttctagttcagaagtaagattttttataaaatctgttaaggaaatttcttagtaacttaaatcttctccgtttgtcgaaatcacttttttgtaccagtcaaagccccgttttttgatacgtttataatctttatctatataaacaaaaccataacgtttttcaaaaccttcacgagtagagtaaaggtccgttgcagaccatgtaagataaccaatcatttctactccctcttcaaccgcttctttcatacgagcaatatggtctgctaaatacttaattctgtaatcatcattaaccgttccg

SEQ ID NO: 7 - DNA, 999 bp, Lactococcus lactis
  CDS (25)..(999): sequence of the L. lactis MG1363 gene whose product is homologous to a formate dehydrogenase
ttcattttatacaaaggagtcccaatgataaaagcaattgccttagaaaatgtttggttaaatttttcagatgaaacaaaagcggctttcaagaaaaataaagcttaccagtttcaatttaaaaaagaagaagagctgacagaatcagattttctggaaacagaagtattagttggtctgccaaagcctgatttattagcaaaatataaaaatttaaaatggctccaacttttatcagctgggaccaatggttatactcaaggagcaaattttcctcaagaggtagttttgacaaatgcaacaggaacttatggacttacgatttctgagcatttactaacaatggctttcgttcttctaagaaaatttgacctttatcaaaaacaacaagaaaaagaaatctgggaaaatattggtcagattcaatctatttatggctcaacagtattggttcatggtttaggtgatattggaagtcactttgcacaaaagattcaagctttgggaggtcatgtcattgcagtcaaacgaactgtttatggtgatgaagaatttgctgatgaagtctatgccgaaactgacctagacaaagttttaccgagagctgatattattgcttcaagtgtccctgggacccatgaaacttataaattatttaatcaagaaaaatttgatttaatgaaagaaaatgctattttcctaaatgttggtcggggaacaaatgtcgatttagaagccttgtgtgatgctcttgagtctaaaaaaattgctggggcaggaattgacgtgaccgacccagaaccattgcctaaaggtcaccgggcttggcatacagaaagactattaatcactcctcatgcttctggcggttatactcttcctgaaacatggcgtcgctttatgaaaatattggaaaaaaatctcgatgcctatgcaaatggtaaggaattgacaaatattgttgatatgaaaacaggatataaacgaaatgctcacaaa

SEQ ID NO: 8 - DNA, 672 bp, Streptococcus thermophilus
  CDS (1)..(672): codY gene fragment
gatattattgattgtaatgctgctattgtaaatggtggaggtgctctccttggttttgctatgaaatacaaaaccaacaatgaccgtgtggaaaagttttttaaagctaaacaacttccagaggaatacatacgtggtatcagccgtgtttatgatactcaagaaaatatcggtattgacagtgacttgaccatcttcccagtggaattaaaagatgatttccctgacggtttgactacaattgcaccaatctatggtggtggtatgcgtcttggttctttcattatttggcgtaacgaccatgattttgtggacgacgaccttatcttggttgagattgcatctacagtagttggtttgcaattgttgcatcttcaaacagaaaacttggaagaaacgattcgtaaacaaacagctattaatatggctattaataccttgtcttactcagaaatcaaggcagtttcagctatcttgaatgagttggacggtttagaaggtcgtttgacagcctctgttatcgcggaccgtatcggaattactcgttctgttattgttaatgctcttcgtaaattagaatcagctggtattattgaaagtcgttcgcttggtatgaaaggcacttacctcaaagtccttaacgaaggtatctacgacaaattgaaagaatac

SEQ ID NO: 9 - DNA, 783 bp, Streptococcus thermophilus
  CDS (1)..(783): full length codY
atggcaaatttgcttgataaaacacgtaaaattacttctatcttgcaacgctcagtagatagtttggaaggagatcttccatacaacaacatggctgctcagttggcagatattattgattgtaatgctgctattgtaaatggtggaggtgctctccttggttttgctatgaaatacaaaaccaacaatgaccgtgtggaaaagttttttaaagctaaacaacttccagaggaatacatacgtggtatcagccgtgtttatgatactcaagaaaatatcggtattgacagtgacttgaccatcttcccagtggaattaaaagatgatttccctgacggtttgactacaattgcaccaatctatggtggtggtatgcgtcttggttctttcattatttggcgtaacgaccatgattttgtggacgacgaccttatcttggttgagattgcatctacagtagttggtttgcaattgttgcatcttcaaacagaaaacttggaagaaacgattcgtaaacaaacagctattaatatggctattaataccttgtcttactcagaaatcaaggcagtttcagctatcttgaatgagttggacggtttagaaggtcgtttgacagcctctgttatcgcggaccgtatcggaattactcgttctgttattgttaatgctcttcgtaaattagaatcagctggtattattgaaagtcgttcgcttggtatgaaaggcacttaccttaaagtccttaacgaaggtatctacgacaaattgaaagaatacgaa

SEQ ID NO: 10 - protein, 259 aa, Lactococcus lactis
LLEKTRKITAILQDGVTDLQQELPYNSMTERLANVIDCNACVINTKGELLGYSLPYNTNNDRVDQFFYDRKLPDEYVRAAVRIYDTMANVPVDRPLAIFPEESLSDFPKGVTTLAPIYGSGMRLGTFIMWREDGEFTDDDLVLVELATTVIGVQLSNLKLEQMEENIRKDTMATMAVNTLSYSEMKAVKAIIEELDGEEGHVIASVIADKIGITRSVIVNALRKLESAGVIESRSLGMKGTYLKVLNTGLFDKLAGRNF

SEQ ID NO: 11 - protein, 463 aa, Lactococcus lactis
MRAILVYYLYALTTADNAGLGLPKAQAMAIVSIYGALVYLSTIVGGWVADRLLGASRTIFLGGILITLGHVALATPFGLSSLFVALFLIILGTGMLKPNISNMVGHLYSKDDSRRDTGFNIFVVGINMGSLIAPLIVGTVGQGVNYHLGFSLAAIGMIFALFAYWYGRLRHFPEIGREPSNPMDAKAKRNFIITLTIVLIVALIGFFLIYQASPANFINNFINVLSIIGIVVPIIYFVMMFTSKKVESDERRKLTAYIPLFLSAIVFWAIEEQSSTIIAVWGESRSNLNPTWFGFTFHIDPSWYQLLNPLFIVLLSPIFVRIWNKLGDRQPSTIVKFGLGLMLTGASYLIMTLPGLLNGTSGRASALWLVLMFAVQMAGELLVSPVGLSVSTKLAPVAFQSQMMAMWFLADSTSQAINAQITPIFKAATEVHFFAITGIIGIIVGIILLIIKKPILKLMGDVR

SEQ ID NO: 12 - protein, 573 aa, Lactococcus lactis
MENVALTHFVDNALRANFIMLHDIDYMVDENQEVLIIDQFTGRTMPGRRYSDGLHQAIEAKEAVPIQDESKTMASITIQNYFRMYKKLSGMTGTAKTEEEEFREIYNIQITPIPTNRPVQRLDHPDLLYPTLEAKFKAVIDDIKRRHAEGQPILIGTVAVETSELISKKLVEAKIPHEVLNAKNHFREAQIIMNAGQQGAVTIATNMAGRGTDIKLGPGVIDHVDPEFRGLAVIGTERHESRRIDNQLRGRSGRQGDPGVSQFYLSLEDELMKRFGSERVSAFLDRMRISGEDAVIKSGLITRQIESSQKRVEGNNYDSRKQVLQYDDVIREQREVIYAQRQEVILATEDMTPVLMGMFKRTIDRQVDGHELAGSLKDEENVKNLLQTLHNTMLPEDGIELSELTGLSVQAMKDLIFDKVKARYASQMEKLSDPERQLEFQRAVILRVVDNNWSEHIDALDQMRQSVGLRGYAQNNPIVEYQEESYKMYNNMIGAIEFEVTRLMMKAQIQPQTAIRQEAPRMTTTASQENITNVDTEHSVSEEISFENVGRNDLCPCGSGKKFKNCHGRTHIA

SEQ ID NO: 13 - protein, 439 aa, Lactococcus lactis
MFFKTLKEAFKVKDVRARILFTIFILFVFRLGAHITVPGVNVQNLTEVSNLPFLNMMNLVSGNAMQNYSLFAMGVSPYITASIIVQLLQMDILPKFVEWSKQGEIGRRKLNQATRYITLVLAMAQSIGITAGFQAMSSLNIVQNPNWQSYLMIGAILTTGSMVVTWMGEQINDQGFGSGVSVIIFAGIVSSIPSAIKSVYDEKFLNVRPSEIPMSWIFVIGLILSAIVIIYVTTFVQQAERKVPIQYTKLTQGAPTSSYFPLRVNPAGVIPVIFAGSITTAPATILQFLQRSQGSNVGWLSTLQNALSYTTWTGMLFYALLIVLFTFFYSFVQVNPEKMAENLQKQGSYIPSVRPGKGTEKYVSRLLMRLATVGSLFLGLISIIPIAAQNVWGLPKIVALGGTSLLILIQVAIQAVKQLEGYLLKRKYAGFMDNPLETK

SEQ ID NO: 14 - protein, 319 aa, Lactococcus lactis
MVNSPQIKQSGSMIFGEQEKVQVVTFMPSEGPTDLHAKIEAAIATFDAEDEVLVLADLWSGSPFNQASAVMGENPERKIAIITGLNLPMLIQAYTERMMDASAGVDKVVANIMKEAKGGIKVLPEELQPAEETAVAAAPAAVQGAIPEGTVIGDGKIKINLARIDSRLLHGQVATAWTPDSRANRIIVVSDTVSKDELRKKLIEQAAPTGVKANVIPIKKMIEVAKDPRFGDTKALLLFETPQDALATIEGGVPIETLNVGSMAHSTGKTMLNKVLSMDKDDVATFEKLRDLGVKFDVRKVPADSKSDLFGLINKADVQ

SEQ ID NO: 15 - protein, 270 aa, Lactococcus lactis
MEYGVLSVILVIVVAFLAGLEGILDQWQFHQPIIACSLIGIVTGHASAGIILGGSLQLIALGWANVGAAVAPDAALASIASSILMVQSNNFDLTHIMGTIVPAAILLATAGLVLTTLVRMLSVVLVHQADRAAENGSYSGVEMWHFIALICQGLRIAIPAGLLLVISPDAIQKALAAIPPVISGGLAVGGGMVVAVGYAMVINLMATREVWPFFFLGFALAPISELTLIATGVLGVVIAIVYLNLQASGGSGNGTASSSGDPIGDILNDY

SEQ ID NO: 16 - protein, 307 aa, Lactococcus lactis
MSENKVTLDKKIRRSVMWRSMFLQGSWNYERMQNGGWAYSLIPALKKLYPSGEEAKEALKRHLEFFNTHPYVAAPIIGVTLALEEERANGADIDDAAIQGVKVGMMGPLAGIGDPVFWFTVRPIVGAIAASLATGGSIIAPLFFFIVWNAIRIAFLWYTQEFGYKSGSAITKDLGGGLLQTVTKGASILGMFVLGVLIQRWVTINFNGPNAVVSKIPLQKGAYLEFPKGSVSGTQLHDILGQVGNKLSLDPTKVTYLQDNLNQLIPGLAGLLITFLCMWLLKKKVSPIVIIFGLFVVGILGRWAQIM

SEQ ID NO: 17 - protein, 325 aa, Lactococcus lactis
MIKAIALENVWLNFSDETKAAFKKNKAYQFQFKKEEELTESDFLETEVLVGLPKPDLLAKYKNLKWLQLLSAGTNGYTQGANFPQEVVLTNATGTYGLTISEHLLTMAFVLLRKFDLYQKQQEKEIWENIGQIQSIYGSTVLVHGLGDIGSHFAQKIQALGGHVIAVKRTVYGDEEFADEVYAETDLDKVLPRADIIASSVPGTHETYKLFNQEKFDLMKENAIFLNVGRGTNVDLEALCDALESKKIAGAGIDVTDPEPLPKGHRAWHTERLLITPHASGGYTLPETWRRFMKILEKNLDAYANGKELTNIVDMKTGYKRNAHK

SEQ ID NO: 18 - protein, 224 aa, Streptococcus thermophilus
DIIDCNAAIVNGGGALLGFAMKYKTNNDRVEKFFKAKQLPEEYIRGISRVYDTQENIGIDSDLTIFPVELKDDFPDGLTTIAPIYGGGMRLGSFIIWRNDHDFVDDDLILVEIASTVVGLQLLHLQTENLEETIRKQTAINMAINTLSYSEIKAVSAILNELDGLEGRLTASVIADRIGITRSVIVNALRKLESAGIIESRSLGMKGTYLKVLNEGIYDKLKEY

SEQ ID NO: 19 - protein, 261 aa, Streptococcus thermophilus
MANLLDKTRKITSILQRSVDSLEGDLPYNNMAAQLADIIDCNAAIVNGGGALLGFAMKYKTNNDRVEKFFKAKQLPEEYIRGISRVYDTQENIGIDSDLTIFPVELKDDFPDGLTTIAPIYGGGMRLGSFIIWRNDHDFVDDDLILVEIASTVVGLQLLHLQTENLEETIRKQTAINMAINTLSYSEIKAVSAILNELDGLEGRLTASVIADRIGITRSVIVNALRKLESAGIIESRSLGMKGTYLKVLNEGIYDKLKEYE

SEQ ID NO: 20 - protein, 259 aa, Lactococcus lactis; identical to SEQ ID NO: 10

SEQ ID NO: 21 - protein, 258 aa, Bacillus subtilis
ALLQKTRIINSMLQAAAGKPVNFKEMAETLRDVIDSNIFVVSRRGKLLGYSINQQIENDRMKKMLEDRQFPEEYTKNLFNVPETSSNLDINSEYTAFPVENRDLFQAGLTTIVPIIGGGERLGTLILSRLQDQFNDDDLILAEYGATVVGMEILREKAEEIEEEARSKAVVQMAISSLSYSELEAIEHIFEELDGNEGLLVASKIADRVGITRSVIVNALRKLESAGVIESRSLGMKGTYIKVLNNKFLIELENLKSH

SEQ ID NO: 22 - DNA, 25 bp, Artificial Sequence
  Description of Artificial Sequence Primer
gggaattctttgggaacaatgataa

SEQ ID NO: 23 - DNA, 23 bp, Artificial Sequence
  Description of Artificial Sequence Primer
cgggatccgttacttctgaacca

SEQ ID NO: 24 - DNA, 21 bp, Artificial Sequence
  Description of Artificial Sequence Synthetic oligonucleotide
tcacctcatataaattcccca

SEQ ID NO: 25 - DNA, 18 bp, Artificial Sequence
  Description of Artificial Sequence Synthetic oligonucleotide
aaatggaacgctcttcgg

SEQ ID NO: 26 - DNA, 24 bp, Artificial Sequence
  Description of Artificial Sequence Synthetic oligonucleotide
cgccagggttttcccagtcacgac

SEQ ID NO: 27 - DNA, 22 bp, Artificial Sequence
  Description of Artificial Sequence Synthetic oligonucleotide
accaacagcgacaataatcaca

SEQ ID NO: 28 - DNA, 21 bp, Artificial Sequence
  Description of Artificial Sequence Primer
cagtatgactgaacgcttggc

SEQ ID NO: 29 - DNA, 21 bp, Artificial Sequence
  Description of Artificial Sequence Primer
gcgataacatgcccttcttca